(19) United States
(12) Patent Application Publication
(10) Pub. No.: US 2010/0120145 A1
(43) Pub. Date: May 13, 2010

(54) THREE-DIMENSIONAL BIOCOMPATIBLE SKELETON STRUCTURE CONTAINING NANOPARTICLES

(75) Inventors: Herwig Brunner, Weilheim (DE); Heike Mertsching, Sindelfingen (DE); Petra Kluger, Gerlingen (DE); Michaela Kaufmann, Schwelm (DE); Achim Weber, Altbach (DE); Gunter Tovar, Waldenbuch (DE); Kirsten Borchers, Stuttgart (DE)

Correspondence Address: HARNESS, DICKEY & PIERCE, P.L.C., P.O. BOX 828, BLOOMFIELD HILLS, MI, 48303, US

(73) Assignee: Fraunhofer-Gresellschaft Zur Forderung Der Angewandten Forschung E.V., Munchen (DE)

(21) Appl. No.: 12/596,825

(22) PCT Filed: Apr. 18, 2008
(86) PCT No.: PCT/EP08/03130

§ 371 Date: Oct. 20, 2009

(30) Foreign Application Priority Data
Apr. 20, 2007 (DE) 10 2007 020 302.2

(51) Int. Cl. C12N 5/073; A61F 2/20; C12N 5/078; B05D 1/12; C12P 1/00; C12N 5/09; A01N 1/00; C12N 5/071; C12N 5/0781; C12N 5/0783; C23C 14/22

(52) U.S. Cl. 435/372.2; 623/9; 435/402; 427/180; 435/41; 435/358; 435/1.1; 435/366; 435/372.3; 204/192.38

(57) ABSTRACT

The invention relates to a cell culture system comprising a three-dimensional, biocompatible frame structure and biocompatible nanoparticles, a method for the cultivation of cells, and the preparation of cells or cell products and tissues per se by means of such a cell culture system.

The present invention relates to a cell culture system comprising a three-dimensional biocompatible framework structure and nanoparticles. The invention further relates to a method for the preparation of such a cell culture system and a method for cultivating cells by means of said cell culture system.

Cells are surrounded in vivo by a complex, dynamic micro-environment consisting particularly of an extracelluar matrix (ECM), growth factors, cytokines, and neighboring cells. The extracellular matrix is present in all four basic tissue types, namely ephithelial, muscular, nerve, and connective and supporting tissue. In addition to their function as a supportive framework, which has long been regarded as their main function, the ECM primarily serves for an interactive signal exchange by means of transmembrane receptors of the cells. Due to this form of communication the same also has an effect on the regulation of gene expression. This plays an important role in essential cell properties, such as adhesion, proliferation, differentiation, migration, apoptosis, and restructuring processes. ECM does not represent a static system, but instead the components of ECM are in a “flow equilibrium” state. Components of ECM are secreted and synthesized by the cells in the intercellular space (the space between the cells), but may also simultaneously be decomposed again by the cells. The extracellular matrix consists of three main components: collagen fibers, anchor proteins, and space-filling carbohydrates. The basal membrane is a protein layer, which may be regarded as the specialized extracellular matrix. It represents a stabilizing layer, and separates the surface epithelia from the connective tissue. This prevents the cells of these layers from sliding apart.

One component of the extracellular matrix is made up of fiber-forming proteins. For this purpose collagens are the predominant protein family of ECM. They form different types of fibers, and are present in almost all tissues. Large parts of ECM are formed by the fibrillar collagen type I, while collagen of the type IV plays an important role in the basal membrane. Elastic fibers are composed of the proteins fibrillin and elastin. Carbohydrates represent an additional important component within ECM. These also include glycosamine oglycane (GAG). Glycosamine oglycanes are proteins associated with long-chained polysaccharides of certain individual modules. If glycosamine oglycanes agglomerate into larger macro-molecules, proteoglycanes are created. Substantial properties and characteristics of ECM are obvious from the plurality and interactions of proteins, glycosamine oglycanes, and proteoglycanes. Further carbohydrate components of ECM are represented by hyaluronic acid, heparane sulfate, chondroitine sulfate, and keratane sulfate.

A further characterizing component of the extracellular matrix includes adhesion proteins. Adhesion proteins, adapter proteins, or other adhesive proteins may interact both with other parts of the matrix, and also with further cells by means of attaching themselves to certain cell receptors. Examples for adhesion proteins are the protein family of laminines, citronektin, and fibronektin.

The basal membrane as a specialized extracellular matrix contains various essential components. First and foremost these include collagens of types Ito IV, wherein type IV has a particularly important meaning. Laminins represent a further part of the basal membrane. They possess a sword-like shape. Their ends are occupied with cell receptors, which mainly bind to integrins. Laminin 1 is the most important adhesion component, and laminin 5 has another special meaning as a key part of the basal membrane. Entactin (nidogen) has a special meaning as a further component of the basal membrane, since the same connects the collagen layers to the laminin layers. Finally, the individual components of the basal membrane are linked by means of proteoglycanes, such as Perlecan, since Perlecan comprises binding sites for collagen, laminin, nidogen, and itself in the structure thereof.

Last but not least, cell receptors should be mentioned as further important elements of the extracellular matrix. Cell membrane proteins, such as the integrin family, play a key role for the cell adhesion in this context. Integrins are heterodimeric proteins, which may be constructed of different alpha and beta sub-units. Depending on the composition of the alpha or beta sub-units of the integrins, the same may bind to the extracellular matrix, such as to laminin, vitronektin, or fibronektin.

One goal for cultivating cells is to reconstruct the extracellular matrix as the natural environment of the cells in a manner that is as accurate as possible. In addition to the parts of the extracellular matrix it is also desirable to be able to incorporate further biologically significant agents, such as growth factors, into said ECM structures.

Currently, the cultivation of primary cells which are isolated from the tissue, often occur in cell culture vessels that are coated with plastic. However, this environment does not correspond to the natural physiological conditions of the cells, and often leads to a loss of function, dedifferentiation of the cells, and in the worst case, to the death of the cells. Currently known improvements of the cell culture vessels consist of coating the surface using individual, natural components of the extracellular matrix, such as collagen fibers or fibronektin. However, the same are not specifically adjusted to the cell type, and are also applied in an undefined manner. The cultivation of cells in gel-like structures containing an active agent is also known. Among others, one disadvantage is that the active agents are washed out in an uncontrolled manner with an optionally necessary change of media.

It is further possible to cultivate cells in cell culture media, in which the active agents are already present in a dissolved form. The active agents are therefore directly available to the cells, however, one disadvantage is that the same are possible used up very quickly, and the concentration thereof cannot be adjusted to the respective differentiation and growth phases of the cultivated cells in a controlled manner.

Nanoparticles and the use thereof for identification and screening methods are known from DE 10144252 A1 and DE 10031859 A1. According to said disclosures, the nanoparticles may also detect or carry certain biologically active substances.

For the preparation of nanoparticles emulsion polymerization represents a special method of polymerization, wherein water-insoluble monomers are emulsified in water by means of emulsifiers, and polymerized using water-soluble initiators, such as potassium persulfate. The polymer dispersions created during the emulsion polymerization, such as latex dispersions, may be utilized for a plurality of applications. U.S. Pat. No. 4,521,317 and U.S. Pat. No. 4,021,364 show the possibilities and limits of emulsion polymerization.

Within the scope of emulsion polymerization two types of emulsions may generally be utilized: the oil-in water emulsion and the water-in-oil emulsion, also known as inverse emulsion. In both cases a monomer that is insoluble in the reaction medium is dispersed by means of the addition of an emulsifier while stirring, and the reaction is initiated by means of adding an initiator.

The present invention is based on the technical problem of providing a cell culture system that comes as close as possible to the native extracellular matrix as the natural environment of the cells, and enables the controlled addition of growth factors and cytokins in order to regulate cell growth and cell differentiation in a controlled manner, and also to adjust the same to commercial requirements. The present invention is further based on the technical problem of providing cells, particularly also tissue and organs, which are suitable for transplants. Furthermore, there is a demand for special tissue within the scope of so-called tissue engineering and for research purposes.

The present invention solves the technical problem by means of providing a cell culture system comprising a three-dimensional biocompatible framework structure and biocompatible nanoparticles, and by means of a method for cultivating cells by means of providing cells or cell products and tissue, and cells or cell products and tissue in and of itself by means of such a cell culture system.

Therefore, the present invention provides a cell culture system which enables the cultivating of cells, particularly eukaryotic cells, and in a particularly preferred embodiment animal or human cells, under conditions corresponding to the in vivo situation, or in a desired synthetically adjusted environment.

The invention is particularly based on providing a cell culture system which together with a three-dimensional biocompatible framework structure has biocompatible nanoparticles present. Such a system enables the subjecting cells cultivated in the same to influences and conditions being specifically provided by the presence of the nanoparticles, and depending on the objective target of cultivation, the influencing of the cells with regard to their biological behavior, i.e. growth or differentiation behavior. In a particularly preferred embodiment the nanoparticles may comprise active agents, for example, in that the same comprise the same in a manner that is enclosed on the surface or in the nanoparticles itself. Of course, it may also be provided that the active agents are present both on the surface and within the interior of the nanoparticles. The active agents being connected to the nanoparticles in this manner are controlled and/or regulated, particularly released in a chronologically delayed manner, within the course of the cell culture method carried out using the cell culture system, and may be added to the cells at such a dose, and over a longer period of time in a controlled manner. In this manner a controlled delivery of said growth factors is ensured over a defined period of time by means of the coupling of the nanoparticles to, for example, growth factors.

It was shown that nanoparticles, for example, made from polymer material, have surprising advantages as a carrier of, i.e. growth factors. Thus, nanoparticles, particularly polymer nanoparticles, as the carrier control the controlled release, e.g. using certain, usually delayed release kinetics, of active agents, such as cytokins. This effect is also called controlled release. A preferred embodiment of the present invention provides that the release of the active agents is regulated in a manner that can be predetermined as a function of time, thus providing desired and specific active agent release kinetics. The invention may provide that desired active agent release kinetics are embodied by means of the selection of the nanoparticles material such that a release of the active agent occurs in a cell-specific manner. For example, a constant release rate of the active agent may occur based on the entire release duration. Of course, the invention may also provide, for example, an initially low active agent release rate in a first phase, and an increased release rate starting after a certain point in time. In a further preferred embodiment the invention also provides that a comparatively high release rate of the active agent is provided in an initial first phase, which is relieved by a low release rate after a certain point in time. By utilizing suitable polymer materials for the preparation of the nanoparticle, it is also possible according to the invention to release a desired high concentration of an active agent from the nanoparticles in the form of a burst effect at a certain point in time within the scope of a cell culture method. In a preferred embodiment the nanoparticles may have such an active agent charge that the active agent concentration is within a preferred range of 1 ng/ml to 10 μg/ml after release.

For this purpose the active agent release may be carried out both by means of diffusion of the agent from or by the nanoparticles, or by means of the decomposition of the nanoparticles itself, for example, by means of hydrolysis of the polymers.

The combination of nanoparticles, particularly those charged with active agents, having a framework structure, particularly made from components of the extracellular matrix, showed further surprising advantages. It is possible to obtain a controlled and aligned differentiation of stem cells by means of cultivating the cells on a framework structure with nanoparticles, particularly of a certain cytokine charge.

It was further shown that particularly primary cells have a significantly higher survival rate by means of cultivation in a matrix, containing nanoparticles, coupled with special growth factors, thus making the long-term cultivation of these sensitive cells possible.

It was surprisingly shown that a controlled and aligned cell migration can be induced by means of cultivation in the cell culture system according to the invention.

Tests show that particularly primary cells, which were cultivated in the cell culture system according to the invention, surprisingly had a significantly increased proliferation. Furthermore, a significantly improved adhesion property to the modified surfaces according to the invention, e.g. the cell culture system, could be determined in comparison to common cell culture vessels. The cells retained their typical morphological properties, even after cultivation over several days. Therefore, the cell culture system according to the invention largely prevents any undesired dedifferentiation of the primary cells.

Within the scope of the present invention the term “cell culture system” means a system for cultivating cells, which provides adhesion locations, particularly in the form of the framework structure contained therein, for cells to be cultivated, preferably in a construction that is structured in a three-dimensional manner, for example, as a matrix or hydrogel. Usually such a cell culture system is positioned in artificial vessels at the time of its use, preferably its in vitro use, which are capable of accommodating the cells to be cultivated via the cell culture system itself, as well as a culture medium.

The cell culture system of the present invention is therefore particularly a system for cultivating cells in vitro. The cell culture system according to the invention may be utilized for cultivating cells in common cell culture vessels, such as Petri dishes or cell culture bottles, or in any other form. In a preferred embodiment of the present invention the cell culture system is utilized for cultivating primary cells. A further embodiment provides that the cell culture system according to the invention is utilized in vivo, i.e. in animal experiments.

In the context of the present invention “framework structure” means a structure for adhering cells. Within the scope of the present invention a preferred embodiment of the framework structure is particularly a structure that not only permits a surface adherence or adherence of cell, but furthermore particularly also a growing in or integrating of the cells into the framework structure itself. The framework structure preferably represents a matrix, preferably having pores or intermediate spaces. In a preferred embodiment of the present invention said matrix has one or more components of the extracellular matrix, such as parts of the basal membrane, adhesion proteins, fiber-forming proteins, carbohydrates, proteoglykanes, or cell receptors.

In the context of the present invention a “primary cell” means a eukaryotic cell of human or animal origin, which is obtained from organs or from an embryo. In particular an embryonic stem cell is preferably provided as a primary cell, preferably such that is obtained from the umbilical cord blood. However, in the context of the present invention the use of human embryonic stem cells is preferably not provided. The stem cell used according to the invention may be a totipotent, omnipotent, or pluripotent cell. Furthermore, the primary cell is preferably an adult stem cell, such an animal or human adult stem cell.

In the context of the present invention “primary cell” also means a eukaryotic cell of human or animal origin. Primary cells may be obtained from organs, such as the skin, kidney, or liver, or from whole embryos. One example for the primary cell used according to the invention is a fibroblast. The primary cell is preferably omnipotent or pluripotent. A primary cell may also be an adult stem cell. The cells are treated by means of the treatment using Trypsin, or by means of another protease, and thus isolated from the tissue, and also separated—by means of the decomposition of intercellular compounds, such as tight junctions. Primary cell cultures of epithelial cells are another example for primary cell. Generally epithelia sheath external and internal surfaces of tissue and organs. Epithelial cells have a very high degree of differentiation which is expressed in the polarization of the cells with an apical and a basolateral surface. The various surfaces assume different functions. For example, the apical surface of epithelial cells of the intestines serves for absorbing nutrients, while the basolateral surface forward said nutrients to the blood, and forms connections to the neighboring cells and to the basal membrane. Primary cells from blood, bone marrow, or spleen, however, may still be cultivated in suspension. After the treatment with Trypsin, additional methods exist in order to further select the isolated sub-type from the isolated primary cells. For example, it is possible to isolate B-cells as the primary cell from, for example, bone marrow, by means of magnetic cell separating methods, or by means of fluorescence activated cell sorting FACS.

A primary cell may preferably also be a non-human embryonic stem cell, preferably an embryonic stem cell that has been obtained from umbilical cord blood in a particularly preferred embodiment. In a preferred embodiment the embryonic stem cell is a cell that has been isolated from an embryo, which is present in a stage of up to eight cells. Said embryonic stem cell from the eight-cell-stage is a totipotent cell, and can be differentiated into all cell forms of the main tissue types, e.g. the endodermal—such as the wall cells of the intestinal tract—, mesodermal—such as muscles, bones, blood cells—and ectodermal—such as skin cells and nerve tissue. In a further preferred embodiment the embryonic stem cell is a cell that has been isolated from an embryo, which is present in the stage of a blastocyst. Said embryonic stem cell from the blastocyst stage is a pluripotent cell, and can be differentiated into all types of body cells of the main tissue types, e.g. endodermal and ectodermal, except for the formation of placenta tissue, which may no longer be formed. A special characteristic of stem cells is that said cells are capable both of self-replicating during cell division, and thus obtaining the pool of stem cells, and simultaneously bringing about a differentiated cell, which in that case has a lower differentiation potential. Stem cells delimit themselves from differentiated cells by means of so-called markers. Markers may be special proteins that are reinforced by stem cells, or are expressed at a low degree. For murine embryonic stem cells SSEA-1, stage-specific embryonic antigen-1, AP activity, alkaline phosphatase activity, and the expression of the transcription factor Oct-3/4 are described as markers. The expression of the marker proteins depends on the origin of the stem cell.

However, the invention also relates to cell culture methods and means for carrying out the same, which relate to, particularly utilize, non-embryonic stem cells. An adult stem cell is a cell that is created after the embryonic stage. Adult stem cells may be isolated from organs and tissue, such as bone marrow, skin, fatty tissue, umbilical cord blood, brain, liver, or the pancreas, and are usually predetermined, e.g. they possess a lower differentiation potential and are multi or unipotent. Usually isolated primary cells from an embryo or an adult mammal have a very limited growth capability. In human fetal cells good cell growth initially occurs after the isolation and cultivation thereof. Furthermore, primary cells may also be isolated and cultivated from tumors. Regulation mechanisms, such as apoptosis, are abrogated by means of genetic changes, oncogenic transformations, or positive growth signals of growth receptors are amplified, respectively. Therefore many, particularly primary tumor cells, often show non-limited growth. A particular sub-population of primary cells under tumor cells represents so-called tumor stem cells. The same were determined as a very small cell fraction within certain tumors. Tumor stem cells have been isolated and cultivated from breast cancer tumors. Characteristic marker proteins for these breast cancer stem cells are a high CD44 expression, no or a low amount of CD24 expression, and the absence of so-called line markers.

In the context of the present invention an “adult stem cell” particularly means a cell that has been created after the embryonic stage, has a differentiation potential that has not yet been exhausted as opposed to the fully differentiated, are predetermined, and are particularly isolated from epithelial cells, endothelial cells, dendritic cells, mesenchymal cells, adipocyten, or particularly the respective precursor cells from heart, skeletal muscles, fatty tissue, skin, and brain.

In the context of the present invention “three-dimensional” means a spatial expansion into all three space coordinates. The expansion may be essentially uniform in these three directions such that, for example, a cylindrical shape, a columnar, cuboidal, or cubic matrix structure is present. However, it is also possible, for example, that the expansion is present in two directions at a greater degree, but in the third direction only at a low degree such that the three-dimensional structure seems plane, for example, illustrating a membrane or a layer.

Within the scope of the present invention the term “biocompatible” means that the framework structure and the nanoparticles causes no toxic, apoptotic, undesired immunological or other undesired reaction both with regard to the material composition thereof, and with regard to the structure thereon in the cells, tissue, or in an organisms, particularly of a trial animal, and does not, or hardly interferes with cellular and molecular processes, even after possible internalization of the nanoparticles, or a decomposition of the nanoparticles and/or framework structure.

In a particular embodiment the invention preferably provides that the nanoparticles are biodegradable, or bioresorbable, e.g. are successively decomposed by means of biological influences, particularly the effect of the cultivated cells, and can release the active agents preferably contained therein.

Within the scope of the present invention nanoparticles are particles having a diameter of 1 to 1000 nm. Such nanoparticles may be composed of different materials, such as inorganic or organic substances. In a preferred embodiment the surfaces thereof may comprise chemically reactive functional groups, which form affine bonds, e.g. covalent and/or non-covalent bonds with complementary functional groups of active agents to be bound, thus being able to reliably fix the active agents onto the surfaces thereof. In another preferred embodiment the present invention provides that the nanoparticles are also able to form bonds with the framework structure. Such bonds are preferably electrostatic interactions.

In a preferred embodiment the present invention provides a cell culture system, wherein the nanoparticles have a diameter of 50 to 1000 nm, preferably of 50 nm to 900 nm, preferably of 60 to 600 nm. In a further preferred embodiment the present invention provides a cell culture system, wherein the nanoparticles have a diameter of 80 to 150 nm, preferably of 50 to 150 nm.

A further embodiment provides that the cell culture system according to the invention contains nanoparticles, which are composed of inorganic substances, such as gold, or other precious metals, or of metals or metal oxides, calcium phosphate, and calcium hydrogen phosphate, or their mixed phosphates, oxidic materials based on silicon, such as silicates, silicon oxides, such as silicon dioxide. In a preferred embodiment the nanoparticles may also be DynaBeads.

A preferred embodiment provides that the cell culture system according to the invention contains nanoparticles, which are comprised of organic materials, particularly organic polymers. Preferably, the nanoparticles may be prepared by means of emulsion polymerization.

Preferably nanoparticles are utilized in the cell culture system according to the invention, which are comprised of biodegradable polymers. Further preferred is also the use of nanoparticles having a diameter of 50 nm and comprising a biodegradable matrix.

A preferred embodiment provides that the cell culture system according to the invention contains nanoparticles, which are comprised of polylactides (PLA), poly(lactide-co-glycolide)s (PLGA), polycaprolactones (PCL), polyglycolides, di- and tri-block polymers, such as PCL/PGA di-block systems, polyorthoesters (POE), polyanhydrides, polyhydroxyalkanoates (PHA), polypyrrolens (PPy), polypropylene carbonate, polyethylene carbonate, polyalkyl cyanonitrile, or polyethylene glycol.

Preferably the invention provides that depending on the desired release profile of the active agent the nanoparticles comprise polymers having a different molecular weight and a variable polarity. The selection of the material to be used for the construction of the nanoparticles may preferably carried out according to in which form and kinetics the active agent is to be released.

The invention particularly provides a preferred embodiment, according to which a nanoparticle is constructed of different materials, particularly of different polymers, in order to obtain a particularly high variability during the control of the release profile of the active agent to be released. Of course, a further embodiment may also provide that the cell culture system according to the invention has nanoparticles comprised of different materials, which in turn have different active agents in a further preferred embodiment. A chronologically and/or spatially specifically defined release of active agents may also take place in this manner.

A particularly preferred embodiment of the present invention also provides that the nanoparticles are carriers of at least one active agent. In the context of the present invention active agents are such agents, which carry out an effect on the cells to be cultivated. Agents preferred as such active agents are those which are involved in the regulation of growth and differentiation processes of the cells to be cultivated. In particular these active agents control, regulate, determine, initiate, or finalize the growth and differentiation processes. Such active agents may also be involved in migration, invasion, redifferentiation or separating activities. In a particularly preferred embodiment the active agents are such which are to be added to the cultivated cells in desired and specific particular application kinetics.

A further preferred embodiment of the present invention provides that the cell culture system contains nanoparticles, which have active agents enclosed in the framework structure and/or on the surface thereof.

A further preferred embodiment of the present invention provides that in order to incorporate active agents, such as growth factors, the water-in-oil technique is utilized as the preferred method for the preparation of nanoparticles charged with active agents, particularly PLA and PLG particles.

For this purpose the present invention preferably also provides a cell culture system, wherein the active agents enclosed in the nanoparticles are growth factors, cytokins, cell adhesion proteins, such as integrins, dyes, such as fluorescenamins, chemokins, vitamins, minerals, fats, proteins, nutrients, fiber-forming proteins, carbohydrates, adhesion proteins, cell receptors, pharmaceuticals, DNA, RNA, aptamers, angiogenic factors, lektins, antibodies, antibody fragments, or inhibitors.

A preferred embodiment of the present invention provides that the cell culture system contains nanoparticles having stabilizers. For this purpose the stabilizers preferably represent carbohydrates, such as trehalose, proteins, polyethylene glycols, or detergents.

A further preferred embodiment of the present invention provides that the cell culture system contains nanoparticles, which have been functionalized by means of coupling to functional groups. A particularly preferred embodiment provides that the nanoparticles themselves comprise functional groups on the surface thereof.

The present invention particularly provides that a first functional group 1A is attached on the surface of the nanoparticles, which is capable of forming an affine, preferably covalent bond with a complementary group 2A of an active agent to be immobilized.

The invention provides that the first functional group 1A is selected from the group consisting of the amino group, carboxy group, epoxy group, maleic mido group, alkyl ketone group, aldehyde group, hydrazine group, hydrazide group, thiol group, and thioester group.

The invention provides that the functional group 2A of the active agent is selected from the group consisting of the amino group, carboxy group, epoxy group, maleic mido group, alkyl ketone group, aldehyde group, hydrazine group, hydrazide group, thiol group, and thioester group. A nanoparticle according to the invention also has a first functional group 1A on the surface thereof, which is covalently linked to a functional group 2A of the active agent to be immobilized, wherein the functional surface group 1A is a different group than the functional protein group 2A. Both groups 1A and 2A forming a bond must be complementary to each other, e.g. capable of forming a covalent bond.

A further preferred embodiment of the invention provides that the surface of the nanoparticle according to the invention has functional groups 1B, and an active agent to be immobilized has the complementary groups 2B binding the functional groups 1B, wherein the functional groups 1B and 2B according to the invention may in particularly form a non-covalent bond.

According to a preferred embodiment of the invention the second functional group 1B of the surface of the nanoparticles is selected from the group consisting of the oligohistidine group, strep-tag I, strep-tag II, desthiobiotin, Biotin, Chitin, Chitin derivatives, chitin binding domain, metal ion chelating complex, streptavidin, streptactin, avidin, and neutravidin.

According to the invention the functional group 2B of an active agent to be immobilized is selected from the group consisting of oligohistidine group, strep-tag I, strep-tag II, desthiobiotin, biotin, chitin, chitin derivatives, chitin binding domain, metal ion chelating complex, streptavidin, streptactin, avidin, and neutravidin. A nanoparticle according to the invention also has a functional group 1B on the surface thereof, which is linked to a functional group 2B of a molecule in a non-covalent manner, wherein the functional surface group 1B of the nanoparticles is a different group than the functional molecule group 2B. Both groups forming a non-covalent bond must be complementary to each other, e.g. capable of forming a non-covalent bond with each other.

Of course, the invention may also provide that the surface of the nanoparticle according to the invention and the active agents to be immobilized optionally have both functional groups 1A and 1B, and 2A and 2B.

A particularly preferred embodiment of the present invention provides that the three-dimensional framework structure contains one or more of the following components, namely fiber-forming proteins, such as collagens, elastic fiber forming fibrillines, and/or elastines, carbohydrates, such as glucosamine glykanes, long-chained polysaccharides, particularly hyaluronic acid, heparane sulfate, chondroitine sulfate, and keratane sulfate, adhesion proteins, such as adapter proteins or other adhesive proteins, such as laminins, vitronektin, and fibronektin, components of the basal membrane, such as laminins, entaktin, and proteoglycanes, and cell receptors for ECM components, such as cell membrane proteins.

A preferred embodiment of the present invention provides that the three-dimensional framework structure contains components of the extracellular matrix, or consists of the same, selected from the group consisting of laminins, clycosamine glykanes (GAG), proteoglykanes, elastin, collagens type I, II, III, and IV, entaktin (nidogene), vitronektin, hyaluronic acid, heparane sulfate, dermatane sulfate, chondroitin sulfate, keratane sulfate, perlecan, adhesion proteins, and fibronektin. In a preferred embodiment of the present invention the framework structure may be constructed of a framework base structure, such as collagen, particularly collagen fibers, wherein said framework base structure is embodied with additional components from the previously mentioned group forming the framework structure in an advantageous and optional manner. In this manner the framework structure, particularly the collagen framework structure, may additionally be embodied, for example, with components promoting the cell characteristics, such as adhesion and proliferation, such as anchor proteins and/or carbohydrates.

In a preferred embodiment of the cell culture system according to the invention the framework structure, particularly made from collagen, is present in fiber form and/or mesh form.

Another preferred embodiment provides that the framework structure, particularly made from collagen, is present in a mesh-like, branched form in a three-dimensional manner. In a preferred embodiment the framework structure may be present in hydro-gel or sponge-like form.

Preferably the three-dimensional biocompatible framework structure in the cell culture system according to the invention represents an extracellular matrix.

A preferred embodiment provides that the concentration of active agents in the nanoparticles is in a range from 1 ng/ml to 10 μg/ml.

A further preferred embodiment of the present invention provides that the cell culture system contains nanoparticles, which are present in the cell culture system in an integrally distributed manner. However, in a preferred embodiment a heterogenous, uneven distribution of the nanoparticles may also be present. In a further preferred embodiment the nanoparticles are present in the form of at least one layer above and/or below the framework structure. In a further preferred embodiment the nanoparticles are present in the form of at least one layer above the framework structure.

Preferably the nanoparticles in the cell culture system according to the invention are arranged in multiple layers below the framework structure.

Preferably the nanoparticles in the cell culture system according to the invention are arranged in multiple layers above the framework structure.

In a preferred embodiment of the present invention the mean diameter of the nanoparticles is always smaller or equal to the mean thickness of the framework structure. In a further preferred embodiment the mean diameter of all nanoparticles is always smaller than the mean thickness of the framework structure. In a preferred embodiment all nanoparticles are preferably embedded, preferably predominantly or completely, in the framework structure. Preferably the ratio of the dimensions of the nanoparticles to the thickness of the framework structure is 1:1 or less, preferably 1:10 or less, further preferably 1:100 or less, further preferably 1:1000 or less.

A preferred embodiment of the present invention provides that the nanoparticles in the cell culture system according to the invention are present in the form of a gradient within the framework structure.

A further embodiment provides that the preferably provided active agent in the cell culture system according to the invention is present in the form of a gradient within the framework structure.

In the context of the present invention the term “gradient” may therefore mean the formation of various concentrations of nanoparticles and/or active agent within the cell culture system according to the invention, particularly the framework structure, particularly a spatially graduated, increasing or decreasing concentration of nanoparticles and/or active agents.

In a preferred embodiment of the present invention an active agent gradient is formed by means of the use of nanoparticles having various concentrations of an active agent. The active agent in this preferred embodiment may be enclosed in or attached to the nanoparticles at various concentrations. Furthermore, the active agent may also be attached to the nanoparticles at various concentrations by means of bonding via functional groups. Depending on the arrangement of said nanoparticles at various concentrations within the framework structure, such as a homogenous distribution, an increasing concentration of a concentration decline of the active agent in the cell culture system may be obtained in this manner. A preferred embodiment of the present invention also provides that an active agent gradient is embodied in that nanoparticles are present within the framework structure in a homogenously distributed manner, wherein the nanoparticles have various active agent concentrations, and are arranged such that an active agent gradient is formed. A preferred embodiment also provides to distribute nanoparticles having various active agent concentrations in a spatially uneven form, e.g. heterogenous, in the framework structure, particularly to incorporate such nanoparticles in the form of a nanoparticle gradient.

A further preferred embodiment of the present invention provides that the active agent gradient is embodied in the cell culture system by means of forming a nanoparticles gradient, namely in that nanoparticles, in which a certain concentration, and the same concentration of an active agent in the nanoparticles utilized is enclosed, or attached to the surface thereof, are present on the framework structure in a spatially heterogenous, e.g. differently distributed, manner. In this manner a low amount of said nanoparticles, for example, in an upper area of the framework structure, followed by a higher amount of said nanoparticles in an area of the framework structure positioned below the same leads to a concentration increase within the framework structure at an incline. Vice versa, a higher amount of said nanoparticles in an upper area of the framework structure, and a lower amount of said nanoparticles in an area of the framework structure positioned below the same leads to a concentration decline within the framework structure at an incline.

Another preferred embodiment of the present invention provides that a controlled and delayed active agent release occurs, particularly by means of the use of biodegradable nanoparticles. The preferably controlled release of active agents according to the invention may be ensured additionally or optionally by means of diffusion from the nanoparticles into the surrounding cell culture system, particularly into the fiber or mesh-like framework structure.

A further preferred embodiment of the present invention provides that the nanoparticles are bonded to the framework structure. In a preferred embodiment the bond is carried out such that a culture medium change does not lead to the separating of the nanoparticles from the framework structure. In a preferred embodiment the bond is rinsable, e.g. the nanoparticles will not be separated from the framework structure, even during a change of the culture medium, and optionally performed rinsing steps utilizing common rinsing media, such as buffers.

Preferably the invention provides that the nanoparticles are bonded to the framework structure via electrostatic interactions, particularly via an ionic bond.

A further preferred embodiment of the present invention provides that the nanoparticles are bonded to the framework structure via UV crosslinking.

Another preferred embodiment of the present invention provides that the cell culture system is utilized for carrying migration tests, particularly migration tests in vivo.

Furthermore, the invention also relates to a method for the preparation of a cell culture system, comprising a three-dimensional biocompatible framework structure, wherein the framework structure is brought into contact with the nanoparticles, for example, according to one of the following methods.

A preferred embodiment of the present teaching provides that the cell culture system is prepared by means of a “contactless printing method.”

In the context of the present invention the term “contactless printing method” is a method, wherein nanoparticles are transferred to the substrate without any contact with the surface. There are different possibilities to achieve this. In a first preferred embodiment a so-called drop on demand is provided by means of an inkjet method. In a second embodiment a method by means of pushbutton or individual pins is provided. In both embodiments one or multiple drops of a suspension are transferred to the desired location. Preferably commercially available machines by Dimatrix—FujiFilm—are utilized for the inkjet method. Further preferred are also machines made by microdrop technologies, MicroFag TECHNOLOGIES, Scienion AG, and GeSIM mbH, which comprise individual pins, and can be regulated for discharging drops via a computer in a point-accurate manner. In the context of the present method “point-accurate” means that the positioning accuracy in all methods is stated at below +/−25 μm. In the context of the positioning accuracy the drop volume should also be taken into consideration. The same may be adjusted to 1 pL in a Dimatix DMC-11601 device, and 100 pL in a Nano-Plotter NP1.2 device by GeSIM. Within the scope of the present invention, the invention preferably provides that the discharge of the nanoparticle suspension is carried out via a pneumatic method, vacuum method, or via a piezoelectric method. The invention provides in particular that the penetration depth of the nanoparticles into the substrate, particularly into the framework structure of the cell culture system, is controlled by the drop volume or the drop speed in the respective embodiments of the method according to the invention. The contactless printing method preferably leads to a layer-like attaching of the nanoparticles within the framework structure.

A cell culture system is provided in a further preferred embodiment of the present application, which is prepared by means of impregnation, particularly in that a framework structure is permeated with a suspension containing nanoparticles, such as by means of saturating with a suspension containing nanoparticles.

A cell culture system is provided in another preferred embodiment of the present invention, which is prepared by means of a laser induced forward transfer LIFT method. For this purpose the invention provides particularly that the material to be transported, particularly one or more parts of the extracellular matrix, is cut out by means of laser energy, and attached to a target material.

Another preferred embodiment of the present invention provides that the cell culture system is prepared by means of electrospinning (spincoating). By means of electrospinning polymer structures may preferably be prepared at a fiber diameter of preferably 2 to 20 μm, which is very similar to the natural environment of the cells, that is to say, of the extracellular matrix.

The invention therefore relates to methods for the preparation of a cell culture system, wherein a particularly preferred embodiment provides to produce the biocompatible nanoparticles, for example, by way of the solvent evaporation of the spontaneous emulsification solvent diffusion (SESD) method, salting out, spray drying, or phase separation.

A particularly preferred embodiment provides to produce the nanoparticles utilized by way of solution evaporation, particularly by way of the water-in-oil-technique. According to this method it is preferably possible according to the invention to also incorporate a variety of hydrophilic active agents into the nanoparticles. For this purpose the active agent located in water is emulsified in an oil phase containing polymer. Said mixture is emulsified in an additional water phase, and the organic solvent is removed, for example, by means of reducing the pressure. According to a further embodiment it is also possible to utilize an oil/water emulsion (O/W), particularly in order to incorporate hydrophobic active agents. In this embodiment the oil phase simultaneously acts both as a solvent for the polymer, and as an active agent.

Another embodiment provides the preparation of the nanoparticles according to the invention particularly by means of the spontaneous emulsification solvent diffusion (SESD) method on the solvent evaporation. For this purpose the invention provides to dissolve both the polymer and the active agent in an organic mixture, preferably in dichloromethane and acetone, and to transfer said solution into an aqueous phase with a stabilizer, and emulsify the same by means of stirring. The invention particularly provides for this purpose that the hydrophilic solvent, preferably acetone, is diffused into the surrounding water phase, and that the nanoparticles according to the invention are created by means of stirring under reduced pressure. A preferred embodiment provides that the particle size is reduced by means of increasing the proportion of solvent that can be mixed in water.

A further embodiment provides to produce the nanoparticles according to the invention particularly by means of salting out. The invention particularly provides to omit the use of solvents in this preparation. For the preparation the polymer, preferably PVA, is added to a saturated solution, particularly magnesium chloride or magnesium acetate, in order to obtain a viscous gel as an aqueous phase in this manner. The invention further provides in particular to dissolve a biodegradable polymer with an active agent, preferably in acetone as the organic phase. The mixing of the aqueous and organic phases provides that the organic phase is emulsified into gel by means of stirring in the two-phase system being created. Preferably the invention provides that the nanoparticles according to the invention precipitate into the aqueous phase by means of adding a sufficient amount of water, and after diffusion of preferably acetone.

Another embodiment provides to produce the nanoparticles according to the invention by means of spray-drying. The invention preferably provides that nanoparticles according to the invention are prepared by means of sputtering or atomizing, respectively, of solutions and emulsions, in which biodegradable polymers and active agents are dissolved, particularly by means of a secondary nozzle in a hot air stream.

A further embodiment provides that the nanoparticles according to the invention are prepared particularly by means of phase separation—coazervation. For this purpose the invention provides to dissolve biodegradable polymers preferably in dichloromethane, and to disperse or emulsify an active agent therein. The invention further provides that preferably silicon oil, which does not mix with the organic polymer phase, is added step-by-step while stirring, and a phase separation is carried out. Said mixture is further stirred while preferably adding heptanes, wherein the nanoparticles according to the invention may be obtained.

The method for the preparation of a cell culture system according to the present invention preferably relates to a cell culture system that is prepared by means of a contactless printing method.

In a further preferred embodiment the present invention relates to a method for the preparation of a cell culture system being prepared by means of impregnation.

In a further preferred embodiment the present invention relates to a method for the preparation of a cell culture system being prepared by means of a LIFT method.

In a further preferred embodiment the present invention relates to the preparation of a cell culture system, wherein the cell culture system is being prepared by means of electrospinning (spincoating).

Furthermore, the present invention also relates to a method for cultivating cells, in that the cells are inserted into a cell culture system according to the present invention and into a cell culture vessel containing a suitable culture medium, and are there cultivated under suitable conditions facilitating cultivation.

Preferably the cultivated cells are eukaryotic cells, particularly of human or animal origin. Preferably the cultivated cells are primary cells.

A further embodiment of the present invention provides that the cell to be cultivated is a tumor cell, particularly a tumor cell line, such as MCF-7, HeLa, HepG2, PC3, CT-26, A125, A549, MRC-5, CHO, MelJuso28, Nalm6, or jurkat.

A further embodiment of the present invention provides that the cell to be cultivated is an adhesive cell, particularly an established cell line, such as HUVEC or HaCaT.

A further embodiment of the present invention relates to a method for cultivating cells by means of the cell culture system according to the invention, wherein the cell to be cultivated is a fully differentiated cell.

In a further preferred embodiment the present invention relates to a method, particularly a non-therapeutic method, for the preparation of differentiated cells or united cell structures made up of non-differentiated cells, wherein the non-differentiated cells are cultivated in the cell culture method according to the invention, and differentiated cells or united cell structures are obtained in this manner.

In a further preferred embodiment the present invention relates to a method, particularly a non-therapeutical method, for maintaining the differentiation stage of pre-differentiated or differentiated cells, wherein the pre-differentiated or differentiated cells are cultivated in the cell culture method according to the invention. In a further preferred embodiment the present invention relates to an above mentioned, preferably non-therapeutical method, wherein non-differentiated cells are cultivated in the cell culture method according to the invention, and are maintained in said non-differentiated state.

Preferably the invention provides that the united cell structures represent transplants or test systems.

The invention further preferably provides that the united cell structures may also be organs, organ parts, particularly a trachea, or parts thereof.

The present invention further relates to differentiated cells and united cell structures, which are obtained after the cultivation according to the invention of differentiated or non-differentiated cells or united cell structures, such as transplants, test systems, organs, organ parts, particularly a trachea, or parts thereof.

The present invention, particularly the present cell culture method, enables the long-term cultivation of stem and precursor cells which may be used for a variety of fields of application, such as for transplants and test systems, but also in fundamental research.

The present invention, particularly the present cell culture method, enables the proliferation of functional cells, particularly of primary cells.

The present invention, particularly the present cell culture method, enables the directional differentiation to functional cells and tissue, such as in the preparation of transplants, or also for test systems in the development of pharmaceuticals.

The present invention, particularly the present cell culture method, enables the preparation of transplants for healing chronic/diabetic wounds, such as by means of cell migration induced by nanoparticles.

Furthermore, the present invention also relates to a method for the preparation of expression products of a cell, wherein the cell can be cultivated according to the cell culture method according to the invention, and the expression product may be obtained from said cell culture.

The present invention also relates to expression products of a cell, which is cultivated according to the method according to the invention, and the expression products of which may be obtained.

Further embodiments are obvious from the sub-claims.

### EXEMPLARY EMBODIMENTS

The present invention is explained in further detail based on the following examples and figures. The examples are to be understood as non-limiting.

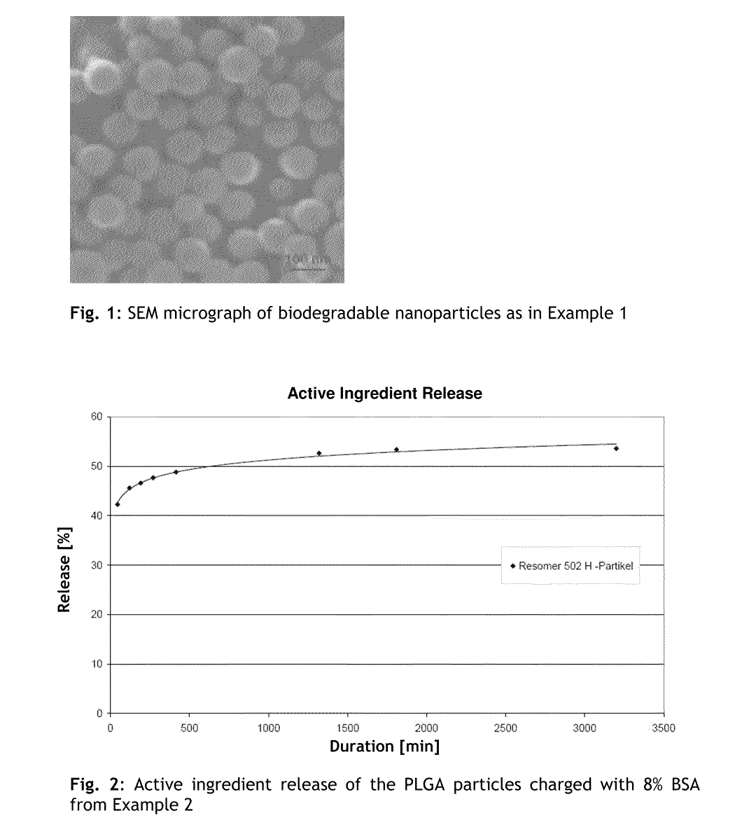

FIG. 1 illustrates an REM image of biodegradable nanoparticles

FIG. 2 illustrates the release result of the PLGA particle charged with 8% BSA from example 1.

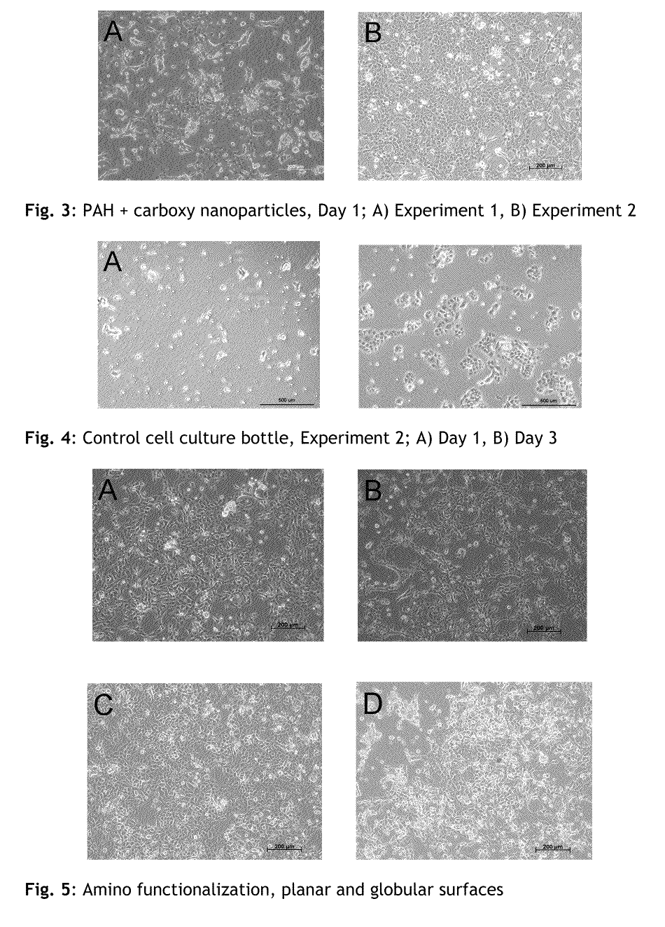

FIG. 3 illustrates cells on an object slide coated with PAH+carboxy nanoparticles on day 1; A) reflects test 1, B) test 2.

FIG. 4 illustrates cells, which have been cultivated in a cell culture bottle as a control, from test 2; A) on day 1, and B) on day 3.

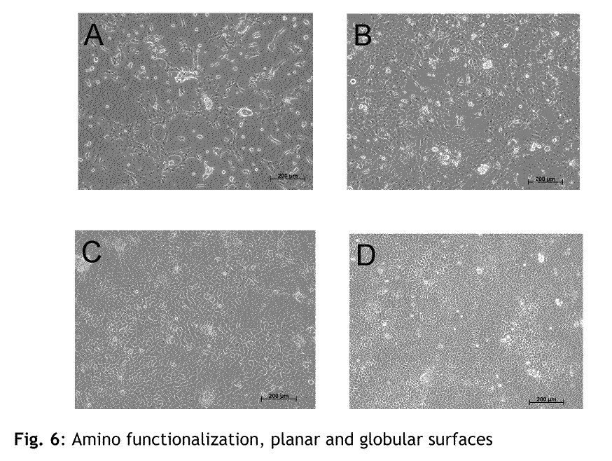

FIG. 5 illustrates cells, which were cultivated on the surfaces using nanoparticles having amino functionalization, and subsequent coating using carboxy nanoparticles having a planar and globular surface; A) PAA, day 1, test 1, B) PAH+carboxy nanoparticles, day 1, test 1, C) PAA, day 3, test 2 D), PAH+carboxy nanoparticles, day 3, test 2

FIG. 6 illustrates cells, which were cultivated on the surfaces using nanoparticles having amino functionalization, and subsequent coating using carboxy nanoparticles having a planar and globular surface; A) PAH, day 1, approach 3, B) PAA+amino nanoparticles, day 1, approach 3, C) PAH, day 3, approach 2, D) PAA+amino nanoparticles, day 3, approach 2

### ABBREVIATIONS

PLGA=poly(lactide-co-glycolide)
PLA=polylactide
PCL=polycaprolactone
PGA=polyglycolide
PAA=polyacrylic acid
PAH=polyallylamine hydrochloride
EGF=epidermal growth factor
BMP=bone morphogenic protein
SESD=spontaneous emulsification solvent diffusion method
MES=morpholine ethane sulfonic acid

### Example 1

### Preparation of Nanoparticles from PLGA Charged with BSA by Means Of Water-in-Oil-in Water Technique (W/O/W)

Initially 120 mg of poly(lactide-co-glycolide) is dissolved in 3.1 mL dichloromethane (O-phase). In this phase an aqueous solution of the protein to be encapsulated (bovine serum albumin, BSA) is emulsified by means of ultrasonic treatment (W/O), in addition 10 mg of BSA was dissolved in 100 μL PBS buffer, pH 7.4. In order to protect the active agent various stabilizers may be incorporated into the aqueous solution, such as sugar, proteins, polyethylene glycols, and other detergents. The W/0 phase prepared is dispersed in a further aqueous phase (100 mL water+500 mg polyvinyl alcohol) by means of ultrasound such that a W/O/W emulsion is created. The organic solution of the 0 phase containing polymer is removed by means of evaporation, and the polymer is thus precipitated as nanoparticles. The size of the particles is approximately 150 to 350 nm. The incorporation of active agent is therefore about 5-8% in weight. A higher charge may also be obtained by means of using a larger amount of active agent.

### Example 2

### Preparation of Biodegradable Nanoparticles from PLA Charged with BMP-2 by Means of W/O/W Technique

10 mg polylactide is dissolved in 300 μL dichloromethane. 10 μL of an aqueous solution of the BMP-2 (bone morphogenic protein-2, c=200 mg/mL) is added to this phase along with 0.05% Lutrol F 68, and then emulsified by means of ultrasound.

Said W1/O phase is dispersed in a second aqueous phase (10 mL of a 0.5% PVA solution) by means of ultrasound. The removal of the dichloromethane/acetone mixture from the W1/O/W2 emulsion is carried out by means of evaporation under vacuum. Nanoparticles of a size of 100-250 nm are precipitated. The active agent charge is approximately 10-15% by weight (FIG. 1).

### Example 3

### Preparation of PLA Nanoparticles Charged with BSA by Means of the W/O/W Method

Initially 120 mg of polylactide-co-glycolide) is dissolved in 3.1 mL dichloromethane/acetone (8/2) (O-phase). In this phase an aqueous solution of the protein to be encapsulated (bovine serum albumin, BSA) is emulsified by means of ultrasonic treatment (W/O), in addition 10 mg of BSA was dissolved in 100 μL water. The W/O phase prepared is dispersed in a further aqueous phase (100 mL water with 0.5% polyvinyl alcohol) by means of ultrasound, and a W/O/W emulsion is created. The organic solution of the O phase containing polymer is removed by means of evaporation, and the polymer is thus precipitated as nanoparticles. The size of the particles is approximately 150 to 300 nm. The incorporation of active agent is therefore about 7-8% in weight.

### Determination of the Release Kinetics

50 mg of the poly(lactide-co-glycolide) particles (Resomer® 502H) charged with BSA are suspended in 5 mL PBS buffer (pH 7.4), and stirred in an oil bath at 37° C. 300 μL is removed per sample lot and centrifuged. The pellet was resuspended in PBS buffer, and returned to the experiment.

The quantitative determination of the active agent released from the particles is carried out by means of different methods, such as HPLC, different protein assays (Lowry or BCA assay), electron spin resonance (ESR), etc. (FIG. 2).

### Example 4

### Coupling of Dyes and Proteins on the Nanoparticles Surface

10 mg of the biodegradable PLGA nanoparticles prepared (from Example 1) are suspended in 0.4 mL 0.1 M MES buffer. 300 μL of a solution of fluoresceinamine (3 mg/mL) is added thereto. However, proteins, such as integrins, may also be bound to the particle surface. 160 μL of an EDC solution (c=110 mg/mL) is added drop-by-drop, and vibrated at room temperature overnight. The suspension is centrifuged, and the pellet is rinsed in a buffer. A linking of 3 μmol of fluoresceinamine per g of particles could be achieved.

### Example 5

### Preparation of Surface Modified Silica Nanoparticles for the Example Using Cells Subsequently being Carried Out

### Synthesis of Silica Particles:

12 mmol tetraethoxy silane and 90 mmol NH₃ is added to 200 ml ethanol. The same is stirred for 24 h at room temperature. Subsequently, the particles are rinsed by means of multiple centrifugations. The result is 650 mg silica particles having a mean particle size of 125 nm.

### Amino Functionalization of Silica Particles:

A 1% by weight aqueous suspension of the silica particles is added to 10% by volume of 25% ammonia. 20% by weight of amino propyl triethoxy silane, based on the particles, is then added, and stirred for 1 h at room temperature. The particles are rinsed by means of multiple centrifugations, and carry functional amino groups on the surfaces thereof (Zeta potential in 0.1 M acetate buffer: +35 mV).

### Carboxy Functionalization of Silica Particles:

10 ml of a 2% by weight suspension of amino functionalized nanoparticles are received in tetrahydrofuran. 260 mg of succinic acid hydride is added. After a 5 minute treatment using ultrasound the same is stirred for 1 h at room temperature. The particles are rinsed by means of multiple centrifugation, and carry functional carboxy groups on the surfaces thereof (Zeta potential in 0.1 M acetate buffer: −35 mV). The mean particle size is 170 nm.

### Example 6

### Optimized Cell Culture Conditions by Means of Nanoparticles Modified Surfaces

Comparative studies are performed on the adhesion, proliferation, migration, and differentiation of primary epidermal cells on the modified surfaces. In addition to the nanoparticles, the modified surfaces also comprise a fiber-like framework structure. The inoculated nanoparticles are present in this fiber-like framework structure at a distance of 10 to 1000 nm.

For the surface modification object slides (OT) are initially rinsed in a water bath at 40° C. using a 2% (v/v) Hellmanex II solution. The subsequent hydroxylation of the surfaces is carried out at 70° C. in a 3:1 (v/v) NH₃ (30%) and H₂O₂ (25%) solution in order to create a negative charge. A coating with, for example, a collagen solution, such as collagen type I, at a concentration of 0.1 to 6 mg/ml is carried out before or after hydroxylation in a generally known manner.

The polyelectrolyte coating of the surfaces is created by means of the so-called layer-by-layer technique. In this method the freshly rinsed and negatively charged OTs are initially placed in a cationic poly(allylamine hydrochloride) solution (PAH solution) (0.01 M; based on the monomer), or poly(diallyl dimethyl ammonium chloride) solution (PDADMAC solution) (0.02 M; based on the monomer), and incubated at room temperature for at least 20 min. The construction of only one cationic PE layer is sufficient for the subsequent coating using the carboxy nanoparticles. For coating using the amino nanoparticles, it is necessary to incubate the OTs after PAH coating for 20 min in an anionic polyacrylic acid solution (PAA solution) (0.01 M; based on the monomer). The linking of the silica nanoparticles is carried out via electrostatic attractive forces. The carboxy nanoparticles are attracted by the cationic polyelectrolyte PAH, and the amino nanoparticles are attracted by the anionic polyelectrolyte PAA. Subsequently the sterilization of the surfaces using 70% ethanol is carried out.

The results of the different surface modifications are illustrated in detail in Table 1. Table 1 shows observations on the morphology of human kreatinocytes on the carboxy and amino nanoparticles. Tests on modified surfaces have been performed in triplicates using n=3 samples each. The colony density, and therefore also the adherent amount of cells and proliferation, as well as the morphology, and therefore the differentiation state of the primary cells, have been assessed. A significantly quicker adhesion and expansion (FIG. 3) of the keratinocytes occurs on the modified surfaces having a fiber-like framework structure as opposed to the culture bottle (FIG. 4). Particularly the surfaces modified with nanoparticles induced a quicker expansion, which is visible after only a few hours.

The morphology of the primary epidermal cells on these modified substrates was comparable to that of the cell culture bottle.

Furthermore, a quicker differentiation of the primary cells was observed on the amino functionalized surfaces (FIG. 6), whereas the differentiation of the keratinocytes on the carboxy surfaces corresponded to those of the control (FIG. 5).

TABLE 1

| | Day 1 | | | Day 2 | | | Day 3 | | |
|---|---|---|---|---|---|---|---|---|---|
| | Test No. | | | | | | | | |
| | 1 | 2 | 3 | 1 | 2 | 3 | 1 | 2 | 3 |
| Glass object | K = + | K = | K = | K = ++ | K = | K = | K = | K = | — |
| slide with PAH | M = + | +(+) | +(+) | M = (+) | ++ | ++ | ++ | ++(+) | |
| | | M = + | M = 0 | | M = + | M = 0 | M = − | M = + | |
| Glass object | K = | K = + | K = | On an | K = | K = | K = | K = | — |
| slide with PAH | ++ | M = + | +(+) | OT* | ++ | ++(+) | ++(+) | ++(+) | |
| and carboxy | M = | | M = 0 | K = | M = + | M = − | M = − | M = 0 | |
| nanoparticles | (+) | | | ++(+) | | | | | |
| | | | | M = + | | | | | |
| | | | | On | | | | | |
| | | | | other | | | | | |
| | | | | OT* | | | | | |
| | | | | M = (0) | | | | | |
| Glass object | K = | K = | K = | K = ++ | K = | K = | K = | K = | — |
| slide with PAA | ++ | ++ | ++ | M = 0 | ++(+) | ++ | ++ | ++(+) | |
| | M = + | M = | M = 0 | | M = | M = 0 | M = − | | |
| | | +(+) | | | +(+) | | | | |
| Glass object | K = | K = | K = | K = ++ | K = | K = | K = | On | — |
| slide with PAA | ++(+) | ++ | ++ | M = (+) | +++ | ++(+) | ++(+) | OT*1 | |
| and amino | M = | M = + | M = + | | M = + | M = + | M = − | M = − | |
| nanoparticle | +(+) | | | | | | | On | |
| | | | | | | | | OT*2 | |
| | | | | | | | | K = | |
| | | | | | | | | +++ | |
| | | | | | | | | M = | |
| | | | | | | | | +(+) | |
| | | | | | | | | On | |
| | | | | | | | | OT*3 | |
| | | | | | | | | M = − | |
| Glass object | K = + | K = | K = + | K = ++ | K = | K = | K = | K = | — |
| slide | M = + | (+) | M = 0 | M = + | ++ | ++ | ++ | ++ | |
| | | M = | | | M = | M = − | M = − | M = + | |
| | | +(+) | | | +(+) | | | | |
| Cell culture | K = 0 | K = + | K = 0 | K = + | K = + | K = + | K = | K = | — |
| bottle (Greiner) | | M = | | M = ++ | M = | M = | ++ | ++ | |
| | | ++ | | | ++ | ++ | M = | M = | |
| | | | | | | | ++ | ++ | |

K = Colony density:
0: no colonies, or less than 5 cells per colony,
+: Colonies < 20 cells per colony,
++: Colonies > 20 cells per colony,
+++: cell layers
M = morphology:
−: cells in a very late differentiation phase, or dead, non-adherent cells,
0: differentiated cells, elongated form, the cytoplasm is large compared to the core,
+: cells in an earlier differentiation phase,
some later differentiated ones ++: all cells in an earlier differentiation phase, cubic form, ratio of cytoplasm/core balanced
*OT = object slide

1. A cell culture system, comprising a three-dimensional, biocompatible frame structure and biocompatible nanoparticles.

2. The cell culture system according to claim 1, wherein the nanoparticles comprise at least one active ingredient.

3-60. (canceled)

61. The cell culture system according to claim 1, wherein the nanoparticles have a diameter of 80 to 150 nm.

62. The cell culture system according to claim 1, wherein the nanoparticles have a diameter of 50 to 150 nm.

63. The cell culture system of claim 1, wherein the nanoparticles have enclosed active ingredients or active ingredients attached to their surface.

64. A method for producing a cell culture system according to claim 1, comprising bringing biocompatible nanoparticles into contact with a three-dimensional, biocompatible frame structure, or producing the cell culture system using a contact-free printing method, or producing the cell culture system by impregnation of a three-dimensional frame structure with nanoparticles, or producing the cell culture system using a LIFT method, or producing the cell culture system by electro-spinning (spin coating).

65. A method for cultivation of cells, comprising placing cells into a cell culture container containing a cell culture system according to claim 1, and cultivating the cells in a cell culture medium under conditions that allow for cultivation.

66. The method according to claim 65, wherein the cells are primary cells.

67. The method according to claim 66, wherein the primary cells are embryonic stem cells.

68. The method according to claim 66, wherein the primary cells are omnipotent or pluripotent stem cells acquired from umbilical cord blood.

69. The method according to claim 66, wherein the primary cells are adult stem cells, in particular epithelial cells, dendritic cells, stromal cells, adipocytes, mesenchymal cells, or bone marrow cells, or wherein the cells are tumor cells, in particular tumor cell lines such as MCF-7, HeLa, HepG2, PC3, CT-26, A125, A549, MRC-5, CHO, MelJuso28, Nalm6 or Jurkat, or wherein the cells are adherent cells, preferably adherent cell lines such as HUVEC or HaCaT, or wherein the cells are differentiated cells.

70. A method for the production of expression products of a cell, comprising cultivating the cell with the method according to claim 65, and obtaining the expression product.

71. A method for the production of differentiated cells or cell clusters, comprising cultivating undifferentiated cells with the method according to claim 65, and obtaining differentiated cells or cell clusters.

72. The method according to claim 71, wherein the cell clusters are transplants, or test systems, or wherein the cell clusters are organs or organ parts, particularly a trachea.

73. An expression product of a cell, produced using the method according to claim 65.

74. Differentiated cells or cell clusters, produced using the method according to claim 65.

75. Transplants or test systems, produced using the method according to claim 65.

76. Organs or organ parts, particularly a trachea, produced using the method according to claim 65.
